SEQUENCE LISTING (11 sequences)

SEQ ID NO: 1 - DNA, 18 bp, unidentified
  source 1..18: mol_type = genomic DNA; note = Bacteriophage T7
taatacgactcactatag

SEQ ID NO: 2 - DNA, 18 bp, unidentified
  source 1..18: mol_type = genomic DNA; note = bateriophage T3
aattaaccctcactaaag

SEQ ID NO: 3 - DNA, 18 bp, unidentified
  source 1..18: mol_type = genomic DNA; note = bateriophage Sp6
atttaggtgacactatag

SEQ ID NO: 4 - DNA, 18 bp, unidentified
  source 1..18: mol_type = genomic DNA; note = bateriophage K11
aattagggcacactatag

SEQ ID NO: 5 - DNA, 18 bp, unidentified
  source 1..18: mol_type = genomic DNA; note = bateriophage gh-1
ttaaaaaccctcactgtg

SEQ ID NO: 6 - DNA, 93 bp, synthetic construct
  source 1..93: mol_type = other DNA
gagcccacactctactcgacagatacgaatatctggacccgaccgtctccactatagtgagtcgtattaggcgtataatacgactcactatag

SEQ ID NO: 7 - DNA, 96 bp, synthetic construct
  source 1..96: mol_type = other DNA
gagcccacactctactcgacagatacgaatatctggacccgaccgtctccactatagtgagtcgtattaggcgtaatttaatacgactcactatag

SEQ ID NO: 8 - DNA, 69 bp, synthetic construct
  source 1..69: mol_type = other DNA
taatacgactcactatagtggagacggtcgggtccagatattcgtatctgtcgagtagagtgtgggctc

SEQ ID NO: 9 - DNA, 69 bp, synthetic construct
  source 1..69: mol_type = other DNA
gagcccacactctactcgacagatacgaatatctggacccgaccgtctccactatagtgagtcgtatta

SEQ ID NO: 10 - DNA, 94 bp, synthetic construct
  source 1..94: mol_type = other DNA
  modified_base 1: mod_base = i
ngagcccacactctactcgacagatacgaatatctggacccgaccgtctccactatagtgagtcgtattaggcgtataatacgactcactatag

SEQ ID NO: 11 - DNA, 97 bp, synthetic construct
  source 1..97: mol_type = other DNA
  modified_base 1: mod_base = i
ngagcccacactctactcgacagatacgaatatctggacccgaccgtctccactatagtgagtcgtattaggcgtaatttaatacgactcactatag

(19) United States
(12) Patent Application Publication
(10) Pub. No.: US 2026/0242838 A1
(43) Pub. Date: Aug. 20, 2026

(54) IN VITRO ENZYMATICAL RNA SYNTHESIS

(71) Applicant: DNA SCRIPT, Le Kremlin-Bicêtre (FR)

(72) Inventors: Tillmann HEINISCH, Le Kremlin-Bicêtre (FR); Guillaume VILLAIN, Le Kremlin-Bicêtre (FR); Benoit DERRIEN, Le Kremlin-Bicêtre (FR); Nelly LOURENCO, Le Kremlin-Bicêtre (FR)

(21) Appl. No.: 19/136,668

(22) PCT Filed: Dec. 8, 2023
(86) PCT No.: PCT/EP2023/084831

§ 371 Date: Jun. 6, 2025

(30) Foreign Application Priority Data
Dec. 9, 2022 (EP) 22212615.3

(51) Int. Cl. C12P 19/34

(52) U.S. Cl. CPC C12P 19/34

(57) ABSTRACT

The present invention relates to nucleic acid constructs capable of generating an RNA product, methods and kits for generating RNA products using said nucleic acid constructs.

The present invention relates to nucleic acid constructs capable of generating an RNA product, methods and kits for generating RNA products using said nucleic acid constructs.

### BACKGROUND

With the development of biotechnology, it is important to be capable of efficiently synthesizing different types of RNA products, such as CRISPR guide RNAs, small RNAs, RNA aptamers, or mRNAs, which are widely used in gene expression or regulation thereof. Among the in vitro RNA synthesis methods already developed, one approach for generating RNA is based on in vitro transcription by using an RNA polymerase and a DNA structure which comprises a double-stranded promoter region and a template for the RNA transcription. Most of the methods of this approach require a complete double-stranded or at least a partial double-stranded DNA structure (Milligan et al., Nucleic Acids Res. 1987 Nov. 11; 15(21):8783-98). A such double-stranded DNA structure is rather time-consuming for preparing, particularly because two independent DNA strands need to be separately synthesized. In addition, none of these template structures allows to produce RNA with accuracy and efficiency.

Therefore, there is still a need to new DNA constructs for generating an RNA product, easy to synthesize and more efficient for in vitro RNA transcription.

### SUMMARY OF THE INVENTION

By working on this problematic, the Inventors have discovered that in vitro RNA transcription can be efficiently implemented by using a nucleic acid structure comprising a hairpin-forming oligonucleotide which comprises successively a reverse complement of a promoter, a loop region and said promoter. Thanks to the loop region, the promoter and its reverse complement are linked in a same oligonucleotide. The nucleic acid structure of the present invention comprises successively, from 5′ to 3′ ends, a single-stranded nucleic acid comprising a template part for generating an RNA to be synthesized, a reverse complement of a promoter, a loop region and said promoter. Therefore, only one single strand of nucleic acid needs to be synthesized by an enzymatic or chemical synthesis process. Surprisingly, the nucleic acid construct of the present invention allows to increase the in vitro transcription efficiency of the template, as compared to the efficiency of in vitro transcription with a complete double-stranded or partial double-stranded DNA structure described in the prior art.

Surprisingly, the Inventors also have discovered that the presence of a termination motif at the 5′end of the template part improves the stop of an RNA polymerase and thus increases the RNA synthesis yield. More surprisingly, the use of a termination motif comprising one or more 2′-O-modified nucleotides further reduces the untemplated activity of an RNA polymerase.

In one aspect, the invention relates to a nucleic acid construct capable of generating at least one RNA product, comprising from 5′ to 3′ (i) a single-stranded nucleic acid comprising at least one template portion for generating the RNA product and (ii) a hairpin-forming oligonucleotide which comprises successively a reverse complement of a promoter, a loop region and the promoter, said reverse complement of a promoter and said promoter being capable of forming a double-stranded oligonucleotide.

In some embodiments, the single-stranded nucleic acid of said nucleic acid construct comprises at least one termination motif at the 5′end of the template part of the single-stranded nucleic acid.

In some embodiments, said termination motif is selected from at least one, preferably at least two, modified nucleotide(s), an abasic site, a hairpin structure, a protein binding site, a chemical linker, or combination thereof. In some embodiments, said termination motif comprises one or two 2′-O-modified nucleotides. For example, the 2′-O-modified nucleotide comprises a 2′-O-modifying group which is 2′-O-(2-methoxyethyl); in such example, the 2′-O-modified nucleotide can be named interchangeably 2′-O-(2-methoxyethyl) nucleotide, 2′-O-(2-methoxyethyl) modified nucleotide, 2′-O-methoxyethyl modified nucleotide or 2′-O-methoxy-ethyl modified nucleotide.

In some embodiments, said nucleic acid construct is a free nucleic acid in solution.

In some embodiments, said nucleic acid construct is attached to a solid support by its 5′ end.

Another aspect of the invention relates to a kit for generating an RNA product, comprising:
an RNA polymerase, ribonucleotides, optionally one or more modified nucleotides, and one or more reaction buffers, and
(i) a nucleic acid construct as described in the present invention, or
(ii) an initiator having a 3′-terminal nucleotide having a free 3′hydroxyl, a template-free DNA polymerase, a plurality of 3′-O-blocked nucleoside triphosphates, a deblocking agent.

In some embodiments, the kit further comprises a solid support, wherein the nucleic acid constructs or the initiators are attached to the solid support by their 5′ end.

In some embodiments, the kit of the invention is a kit for generating a single-guide RNA (sgRNA), said kit comprises an initiator having a deoxyribonucleic acid sequence complementary to a nucleic acid sequence of a constant part of a sgRNA.

In some embodiments, the kit for generating a sgRNA comprises an RNA polymerase, several ribonucleotides and a nucleic acid construct of the invention comprising a template part for generating a sgRNA.

Another aspect of the invention relates to a method of generating an RNA product, comprising the step of:
(a) providing a nucleic acid construct as described in the present invention,
(b) contacting the nucleic acid construct with an RNA polymerase and ribonucleotides to produce the RNA product,
(c) optionally repeating step (b),
(d) optionally implementing at least one post-transcriptional modification,
(e) optionally recovering said RNA product produced in steps (b), (c) and/or (d).

### BRIEF DESCRIPTIONS OF THE DRAWINGS

FIG. 1A diagrammatically illustrates an embodiment of a nucleic acid construct of the invention, which comprises, from 5′ end to 3′ end, a single-stranded nucleic acid comprising a template part for generating an RNA product, a reverse complement (RC) of a promoter, a loop region and said promoter.

FIG. 1B diagrammatically illustrates another embodiment of a nucleic acid construct of the present invention, which is attached to a solid support by its 5′ end and comprises, from 5′ end to 3′ end, a single-stranded nucleic acid which comprises a termination motif and a template part for generating an RNA product, a reverse complement (RC) of a promoter, a loop region and said promoter.

FIG. 1C diagrammatically illustrates an embodiment of a nucleic acid construct of the invention, according to FIG. 1B, wherein the template part corresponds to sequences for generating a sgRNA.

FIG. 1D diagrammatically illustrates that a sgRNA product is generated by in vitro transcription of a nucleic acid construct of the invention illustrated in FIG. 1C.

FIG. 2 shows the levels of the fluorescence produced by Broccoli fluorescent RNAs, which were respectively generated by in vitro transcription from five free nucleic acid constructs, i.e. two constructs of the invention named “6-nt loop” and “9-nt loop”, two constructs of the prior art as positive control named “dsDNA” and “ssDNA+promotor”, and a single stranded construct as negative control named “ssDNA”. The fluorescence was measured during 12 hours of in vitro transcription.

FIG. 3A shows the levels of the fluorescence produced by Broccoli fluorescent RNAs, which were respectively generated by in vitro transcription from six nucleic acid constructs, i.e. two free constructs of the invention (named “6-nt loop” and “9-nt loop”) and two constructs of the invention immobilized on a resin (named “Resin 6-nt loop” and “Resin 9-nt loop”), a construct of the prior art “dsDNA” as positive control and a single stranded construct “ssDNA” as negative control. The fluorescence was measured during 18 hours of in vitro transcription.

FIG. 3B shows electrophoresis data of the Broccoli fluorescent RNA generated by in vitro transcription from four constructs of the invention, i.e. “6-nt loop”, “9-nt loop”, “Resin 6-nt loop”, “Resin 9-nt loop”, and from the construct “dsDNA” as positive control.

FIG. 4 shows electrophoresis data of RNAs generated by in vitro transcription from 6 constructs of the invention. Said constructs comprise a single-stranded nucleic acid which is composed of, from 5′ to 3′, a sequence of 15 nucleotides, a termination motif, and a sequence of 21 nucleotides as the template part for generating an RNA product. The termination motifs in these constructs are at the 5′ end of the sequence of 21 nucleotides. The tested termination motifs are AP2 (two adjacent abasic sites), AP3 (three adjacent abasic sites), APiAP (two abasic sites which are separated by a deoxyinosine), APTAP (two abasic sites which are separated by a deoxythymine), APT3 (three abasic sites each being separated by a deoxythymine) or C9S (or C9SP, also called Spacer 9 which is named 9-O-Dimethoxytrityl-triethylene glycol, 1-[(2-cyanoethyl)-(N,N-diisopropyl)]-phosphoramidite, CAS #146668-73-7). The C9S is thus a triethylene glycol spacer. The constructs “IVT Stop” and “No stop” are two constructs used as control. “IVT stop” construct comprises a single-stranded nucleic acid of 21 nucleotides as template. “No stop” construct comprises a single-stranded nucleic acid comprising a sequence of 21 nucleotides directly followed by a sequence of 15 nucleotides (i.e., no termination motif is positioned in-between the two sequences).

FIG. 5A shows the percentage of perfect sgRNA sequences generated from constructs of the invention, which comprise a template part for generating a sgRNA and a termination motif at the 5′ end of said template part. The tested termination motif is either AP2, C9S, dU2 (deoxyuridine, which is converted to an abasic site after the treatment by UDG enzyme), or AP2/C9S combined with a 2′-O-methoxy-ethyl modified nucleotide (“C9S-Methox” and “AP2-Methox”). For each sgRNA, the percentages of perfect sequence (Perfect, dark), perfect sequence with 1 additional dT (Perfect T, light grey) and perfect sequence with 2 additional dT (Perfect TT, dark grey) are indicated.

FIG. 5B shows electrophoresis data of the RNAs generated by in vitro transcription from 4 constructs of the invention. Said constructs comprise a single-stranded nucleic acid which is composed of, from 5′ to 3′, a sequence of 15 nucleotides, a termination motif, and a sequence of 21 nucleotides as the template part for generating an RNA product. The termination motifs in these constructs are at the 5′ end of the sequence of 21 nucleotides. The tested termination motifs are C9SP alone (No Met) or C9SP combined with one (1Met) or two (2 Met) 2′-O-methoxy-ethyl modified nucleotides. The constructs “IVT Stop” and “No stop” as described in FIG. 4 are used as controls.

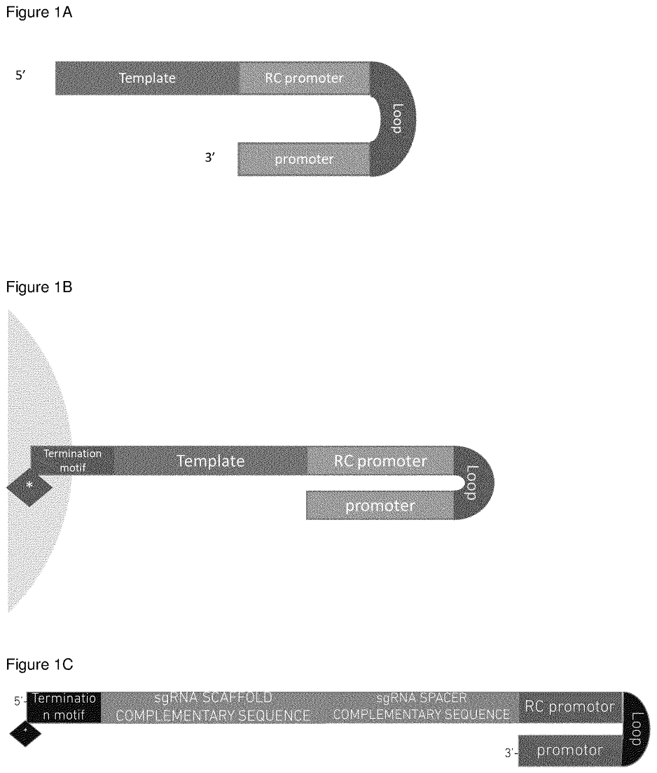

### DETAILED DESCRIPTION OF THE INVENTION

### Definitions

The present disclosure will be best understood by reference to the following definitions.

In the context of the present invention, the term “template part” refers to a part of a single-stranded nucleic acid which may be transcribed by an RNA polymerase for producing an RNA product through base pairing.

The term “promoter” as used herein refers to a single-stranded DNA comprising a sequence of nucleotides that is involved in recognition and binding of an RNA polymerase and/or other proteins such as transcriptional factors which are necessary for initiating the transcription of a gene. A promoter is a region of DNA where RNA polymerase begins to transcribe a gene. Normally, promoter sequences are typically located directly upstream or at the 5′ end of the transcription initiation site (Lin et al., 2018 Molecular Cell, Volume 70, issue 1, P60-71). Both promoters and transcription initiation sites are bound by RNA polymerase and the necessary transcription factors. Promoter sequences describe the direction of transcription and point out which DNA strand will be transcribed.

The term “reverse complement” as used herein refers to a sequence of nucleotides within a strand of nucleic acid which is complementary, but in the reverse order, to a given sequence of nucleotides within the same strand of nucleic acid. Thereby, when the single strand nucleic acid folds back on itself, the given sequence of nucleotides and its reverse complement can hybridize to one another, to form a double-stranded region.

The term “hairpin-forming oligonucleotide” as used herein refers to an oligonucleotide, which comprises a first sequence of nucleotides, a second sequence of nucleotides that is a reverse complement of said first sequence of nucleotides and a non-self-complementary central region which connects said first sequence of nucleotides and said second sequence of nucleotides. The first sequence of nucleotides and the second sequence of nucleotides are able to hybridize under adequate DNA hybridization conditions to form a double-stranded region. In this conformation, the non-self-complementary central region forms a single-stranded loop and a hairpin comprising a loop region is formed.

As used herein, the term “complementary” in the context of the nucleotides refers to the base pairing between two nucleotides and/or the stacking of bases. The base-pairing may be direct or reverse Hoogsteen base pairing, or direct or reverse Watson & Crick base pairing. Complementary nucleotides may be A and T (or A and U), or C and G. Complementarity may also exist between other natural or non-natural nucleotides if hydrogen bonds may be formed between two nucleotides. The term “complementary” in the context of nucleic acid refers to the ability of two single-stranded nucleic acids to form an anti-parallel, double-stranded nucleic acid structure. Two single-stranded nucleic acids which are substantially complementary will hybridize to each other under DNA hybridization conditions. The term “substantially complementary” refers both to complete complementarity of two nucleic acids as well as complementarity sufficient to achieve the desired binding of two nucleic acid. DNA hybridization conditions are well known in the art and defined by salt concentrations and hybridization temperature.

### Nucleic Acid Construct

The present invention relates to a nucleic acid construct that may be used to generate an RNA product. Said nucleic acid construct comprises from 5′ to 3′ (i) a single-stranded nucleic acid comprising a template part for generating an RNA product and (ii) a hairpin-forming oligonucleotide which comprises successively a reverse complement of a promoter, a loop region and said promoter.

The nucleic acid construct of the invention has a linear primary structure, i.e. said nucleic acid construct is a primary single-stranded nucleic acid. However, the nucleic acid construct of the invention is able to form locally a hairpin secondary structure on its 3′ end, thanks to the complementarity between a first sequence of nucleotides which is a promoter and a second sequence of nucleotides which is the reverse complement of the promoter and a non-self-complementary central region as a loop region which connects the promoter and its reverse complement. Thus, under adequate conditions, the nucleic acid construct of the invention comprises a single-stranded nucleic acid comprising a template part for generating an RNA product and a double-stranded region, which is formed by the promoter and its reverse complement.

Thus, the nucleic acid construct of the invention provides a more efficient and easier solution for generating an RNA product.

### Single-Stranded Nucleic Acid

According to the invention, the length and the sequence of the single-stranded nucleic acid in the nucleic acid construct may be determined by the RNA product to be generated. Said single-stranded nucleic acid may comprise any number of nucleotides. According to an embodiment, said single-stranded nucleic acid comprises at least 10 nucleotides. The single-stranded nucleic acid may typically comprise from 10 to 10000 nucleotides, particularly from 10 to 5000 nucleotides, more particularly from 10 to 3000 nucleotides, from 10 to 1000 nucleotides, from 10 to 800 nucleotides, from 10 to 500 nucleotides, still more particularly from 10 to 300 nucleotides. Generally speaking, a mRNA may comprise up to 10000 nucleotides, in particular from 100 to 5000 nucleotides. Small RNAs may comprise up to 250 nucleotides. For examples, small interfering RNAs (siRNAs) and microRNAs (miRNAs) may respectively comprise from 20 to 30 and from 20 to 50 nucleotides. The length of an average small nuclear RNAs (snRNA) is around 150 nucleotides.

Said single-stranded nucleic acid may comprise natural deoxyribonucleotides and/or non-natural nucleotides. Natural deoxyribonucleotides are, for examples, deoxyadenosine, deoxycytidine, deoxyguanosine, and deoxythymidine. Non-natural nucleotide may include modified bases, sugars, or internucleosidic linkages. For instance, non-natural nucleotides may comprise phosphorothioate internucleosidic linkages, bases containing linking groups permitting the attachment of labels, such as fluorophores, or haptens, and the like. Examples of non-natural nucleotide may be 2′-O-methyl modified nucleotides, 2′-F modified nucleotides, alpha-phosphothioate modified nucleotides, or 2′-O-methoxy-ethyl modified nucleotides.

In some embodiments, said single-stranded nucleic acid comprises a template part for generating an RNA product. The template part is the part of said single-stranded nucleic acid which is transcribed by an RNA polymerase for generating an RNA product.

In some embodiments, the sequence of the template part of the single-stranded nucleic acid is complementary to the sequence of an RNA product to be generated. A skilled person is able to adapt the sequence of the template part of the single-stranded nucleic acid according to the sequence of the RNA product to be generated.

In some embodiments, said single-stranded nucleic acid may comprise the template part for generating a single-guide RNA (sgRNA), a small RNA, an RNA aptamer or a mRNA.

In some embodiments, said single-stranded nucleic acid may consist of the template part for generating a single-guide RNA (sgRNA), a small RNA, an RNA aptamer or a mRNA.

In some embodiments, said single-stranded nucleic acid may consist of the template part for generating a single-guide RNA (sgRNA), a small RNA, an RNA aptamer or a mRNA and of at least one termination motifs at the 5′end.

The term “mRNA” refers to a single-stranded RNA which can be read by a ribosome in the process of synthesizing a polypeptide or a protein. In some embodiments, an mRNA may comprise up to 10000 nucleotides.

The term “single-guide RNA” as used herein refers to a single-stranded RNA which may be used in CRISPR/Cas9-mediated gene editing. Said single-guide RNA comprises from 5′ to 3′ a variable part whose sequence is target specific and a constant part whose sequence is recognized by Cas 9 nuclease and constant for sgRNAs.

The term “small RNA” as used herein refers to a non-coding single-stranded RNA of less than 250 nucleotides. Examples of small RNAs include, but not limited to, small interfering RNAs (siRNA), microRNAs (miRNA), mitochondrial RNA (mtRNA), small hairpin RNA (shRNA), piwi-interfering RNAs (piRNA), small nuclear RNAs (snRNA), small nucleolar RNAs (snoRNA), and small rDNA-derived RNAs (srRNA).

The term “RNA aptamer” refers to a single-stranded RNA that can selectively bind to a specific target, including proteins, peptides, carbohydrates, small molecules, toxins, and even live cells. Examples of RNA aptamers are fluorescent light-up RNA aptamers which can selectively bind to a fluorophore and then enhance its fluorescence.

In some embodiments, the single-stranded nucleic acid in a nucleic acid construct of the invention comprises the template for generating an RNA product selected from a sgRNA, a miRNA, a mtRNA, a siRNA, a shRNA, and an RNA aptamer.

In some embodiments, the single-stranded nucleic acid in a nucleic acid construct of the invention consists of the template for generating an RNA product selected from a sgRNA, a miRNA, a mtRNA, a siRNA, a shRNA, and an RNA aptamer and optionally at least one, termination motif at the 5′end.

In some embodiments, the single-stranded nucleic acid comprises a the template for generating a sgRNA and comprises from 5′ to 3′ a nucleic acid sequence complementary to a nucleic acid sequence of the constant part of a sgRNA and a nucleic acid sequence complementary to a nucleic acid sequence of the variable part of the sgRNA. A skilled person is able to design the sequence complementary to the variable part of a sgRNA according to the target to be edited by using any conventional methods.

In some embodiments, the single-stranded nucleic acid consists of the template for generating a sgRNA and comprises from 5′ to 3′ a nucleic acid sequence complementary to a nucleic acid sequence of the constant part of a sgRNA and a nucleic acid sequence complementary to a nucleic acid sequence of the variable part of the sgRNA and optionally at least one termination motif at the 5′end. A skilled person is able to design the sequence complementary to the variable part of a sgRNA according to the target to be edited by using any conventional methods.

According to the invention, said single-stranded nucleic acid may consist of a template part for generating an RNA product and optionally at least one termination motif at the 5′end of the template part of said single-stranded nucleic acid.

### Termination Motif

Surprisingly, the Inventors have shown that the presence of a termination motif, especially a termination motif comprising one or more O-methoxy-ethyl modified nucleotides, at the 5′end of the template part may improve the release of an RNA polymerase from the generated RNA product and thus increases the RNA synthesis yield. In a preferred embodiment, the termination motif comprises at least two, three, four or more modified nucleotides, in particular two, three, four or more O-methoxy-ethyl modified nucleotides.

Accordingly, the single-stranded nucleic acid of the invention may further comprise at least a termination motif at the 5′ end of the template part of said single-stranded nucleic acid, i.e. said single-stranded nucleic acid comprises at least a termination motif and a template part for generating an RNA product, wherein the termination motif is at the 5′ end of said template part.

The term “termination motif” refers to a motif that promotes the release of an RNA polymerase from the DNA template to stop the transcription. Generally speaking, a termination motif is a nucleic acid having a specific sequence which destabilizes the affinity between DNA template and an RNA polymerase which is bound to the DNA template. Said termination motif may be any naturally occurring procaryotic, eucaryotic or viral termination motif known in the art, e.g. the termination motifs isolated from or derived from bacteria or bacteriophages, the variants thereof, or any non-natural termination motif which has been described in the art, e.g. an abasic site or a modified nucleotide.

In some embodiments, said single-stranded nucleic acid comprises, from 5′ to 3′, a part of nucleic acid which is not transcribed to RNA, a termination motif, and a template part for generating an RNA product, wherein the termination motif is at the 5′ end of said template part. The part of nucleic acid non-transcribed may comprise, for example, a cleavable motif.

In some embodiments, said single-stranded nucleic acid consists of a termination motif and a template part for generating an RNA product, wherein the termination motif is at the 5′ end of said template part. A single-stranded nucleic acid may comprise more than one termination motif, e.g. two termination motifs. In a particular embodiment, said single-stranded nucleic acid comprises at least two termination motifs.

In some embodiments, the termination motif is selected from at least one modified nucleotide, an abasic site, a hairpin structure, a protein binding site, a chemical linker, and combinations thereof. In a particular embodiment, the termination motif comprises two or more components selected from modified nucleotides, abasic sites, a hairpin structure, protein binding sites, chemical linkers, and combinations thereof. For instance, the termination motif comprises two or more modified nucleotides, and optionally one or more chemical linker or spacer. In a particular embodiment, the termination motif comprises at least one modified nucleotide and at least one component selected from at least one modified nucleotide, an abasic site, a modified abasic site, a hairpin structure, a protein binding site, a chemical linker and combinations thereof. In a particular embodiment, the termination motif comprises at least two modified nucleotides and at least one component selected from an abasic site, a modified abasic site, a hairpin structure, a protein binding site, a chemical linker and combinations thereof.

Examples of modified nucleotides which can be cited as a termination motif are 5-octadiynyl dU, 6-carboxyfluorescein ddI, 1,2′-dideoxyriboseI, a 2′O-methyl modified nucleotide, a 2′-O-methoxy-ethyl modified nucleotide or a deoxynucleotide with a bulky side chain.

In some embodiments, the termination motif comprises a 2′-O-methoxy-ethyl modified nucleotide, e.g. 2′-O-methoxy-ethyl modified AMP, 2′-O-methoxy-ethyl modified CMP, 2′-O-methoxy-ethyl modified GMP, or 2′-O-methoxy-ethyl modified UMP.

In some embodiments, the termination motif is chosen from the group consisting in AP1, AP2, AP2-Methox, AP3, APiAP, APTAP, APT3, C3S, C9S, C9S-Methox, dU2, and at least one of their combinations.

In some embodiments, the termination motif consists of a 2′-O-methoxy-ethyl modified nucleotide, e.g. 2′-O-methoxy-ethyl modified AMP, 2′-O-methoxy-ethyl modified CMP, 2′-O-methoxy-ethyl modified GMP, or 2′-O-methoxy-ethyl modified UMP. In some embodiments, the termination motif consists of two, three, four or more 2′-O-methoxy-ethyl modified nucleotides.

The term “abasic site”, abbreviated “AP”, as used herein refers to a deoxyribose in a DNA strand that has no nucleobase. Therefore, in the context of the invention, an abasic site is a position in the nucleic acid construct that has deoxyribose without a purine base or a pyrimidine base.

In some embodiments, the termination motif may comprise one or more adjacent abasic sites or more than one abasic sites which are separated one to another by a nucleotide, e.g. a deoxyinosine or a deoxythymine.

In some embodiments, the termination motif comprises of an abasic site. In some embodiments, the termination motif consists of an abasic site.

In another embodiment, the termination motif comprises two or three adjacent abasic sites. In another embodiment, the termination motif consists of two or three adjacent abasic sites.

In some embodiments, the termination motif comprises two abasic sites which are separated by a nucleotide. In some embodiments, the termination motif consists of two abasic sites which are separated by a nucleotide.

In another embodiment, the termination motif comprises three abasic sites which are separated one to another by a nucleotide. In another embodiment, the termination motif consists of three abasic sites which are separated one to another by a nucleotide.

In some embodiments, the termination motif comprises one or more deoxyuridines (dU) which may be converted to an abasic site, e.g. by the treatment of UDG enzyme.

In some embodiments, the termination motif consists of two abasic sites which are separated by a deoxyinosine. In some embodiments, the termination motif consists of two abasic sites which are separated by a deoxythymine. In some embodiments, the termination motif consists of three abasic sites which are separated one to another by a deoxythymine.

In some embodiments, the termination motif comprises or consists of (i) one or more modified nucleotides and (ii) one or more abasic sites. For instance, the termination motif comprises or consists of (i) two modified nucleotides and (ii) one abasic site, wherein the abasic site is inserted between the two modified nucleotides. In some embodiments, the termination motif may comprise or consist of (i) one or two 2′-O-methoxy-ethyl modified nucleotides and (ii) two adjacent abasic sites which are combined to the 5′ end of said modified nucleotide(s). In some embodiments, the termination motif may comprise or consist of two 2′-O-methoxy-ethyl modified nucleotides separated by one abasic site.

In some embodiments, a termination motif may also be a chemical linker. In some embodiments, the chemical linker is a triethylene glycol spacer or a phosphoramidite spacer, such as C3S (or C3SP, also known as C3 spacer or C3 spacer phosphoramidite which is 3-(4,4′-Dimethoxytrityloxy) propyl-1-[(2-cyanoethyl)-(N,N-diisopropyl)]-phosphoramidite) or C9S.

In some embodiments, the termination motif comprises or consists of (i) one or more modified nucleotides and (ii) one or more chemical linkers. Preferentially, the termination motif comprises or consists of (i) at least two modified nucleotides and (ii) one or more chemical linkers. Preferentially, an chemical linker is inserted between two modified nucleotides. In some embodiments, the termination motif comprises or consists of (i) two modified nucleotides and (ii) one chemical linker. In some embodiments, the termination motif consists of two modified nucleotides separated by one chemical linker. In some embodiments, the termination motif may comprise (i) one or two 2′-O-methoxy-ethyl modified nucleotides and (ii) a triethylene glycol spacer which are combined to the 5′ end of said modified nucleotide(s). In some embodiments, the termination motif comprises two 2′-O-methoxy-ethyl modified nucleotides separated by the chemical linker C3S. In some embodiments, the termination motif comprises two 2′-O-methoxy-ethyl modified nucleotides separated by the chemical linker C9S.

The term “hairpin structure” when referring to a termination motif refers to a sequence of nucleotides that may have a secondary hairpin structure. Some bacterial intrinsic terminators may have a hairpin structure. Examples of these types of termination motifs include Rho-independent terminators, more particularly T7 terminator.

The term “protein binding site” as used herein refers to a deoxyribonucleic acid whose complementary RNA can be recognized by a protein involved in the release of an RNA polymerase from the generated RNA. Examples of these termination motifs include Rho-dependent terminators whose complementary RNA can be recognized by Rho factor.

In some embodiments, the termination motif is a termination motif isolated from or derived from a bacterium or a bacteriophage, such as T7 Terminator or any variants thereof. Such variants have been described in the art (Mairhofer et al., ACS Synth Biol. 2015 Mar. 20; 4(3):265-73; Macdonald et al., J Mol Biol. 1994 Apr. 29; 238(2):145-58.) or may be determined by routine methods. In some embodiments, the nucleic acid construct does not comprise any termination motif.

### Hairpin-Forming Oligonucleotide

In some embodiments, the single-stranded nucleic acid is located proximate 5′ end of a hairpin-forming oligonucleotide, i.e., within the limit of 5, 4, 3, 2 or 1 nucleotide(s) before the 5′ end of said hairpin-forming oligonucleotide, or exactly 5′ end of said hairpin-forming oligonucleotide. In a particular embodiment, the 3′ end of the single-stranded nucleic acid is directly connected to the 5′ end of the hairpin-forming oligonucleotide, i.e., directly connected to the 5′ end of the reverse complement of a promoter.

In some embodiments, the hairpin-forming oligonucleotide successively comprises, from 5′ to 3′, a reverse complement of a promoter, a loop region and said promoter. In other words, the hairpin forming oligonucleotide comprises from 3′ to 5′ a promoter, a reverse complement of the promoter and a loop region in-between, which connects the promoter and the reverse complement of the promoter.

The loop region is the connecting part between the promoter and its reverse complement. Said loop region is formed by a single-stranded oligonucleotide, which is composed of natural and/or non-natural deoxyribonucleotides and/or ribonucleotides. Natural deoxyribonucleotides are, for examples, deoxyadenosine, deoxycytidine, deoxyguanosine, and deoxythymidine. Natural ribonucleotides are, for examples, adenosine, cytidine, guanosine, and uridine. Non-natural deoxyribonucleotides and ribonucleotide may include modified bases, sugars, or internucleosidic linkages. Advantageously, the sequence of the loop region is not complementary with any other parts of the nucleic acid construct of the invention.

Surprisingly, the Inventors have shown that the presence of a loop region to bind the promoter and its reverse complement in the nucleic acid construct, particularly a loop region having from 6 to 9 nucleotides, may improve the transcription efficiency compared to transcription efficiency by use of a double-stranded DNA structure or a partial double-stranded DNA structure of the prior art.

In some embodiments, the loop region comprises at least 3 nucleotides, in particular from 4 to 15 nucleotides, more particularly from 5 to 10 nucleotides. In a particular embodiment, the loop region comprises or consists of 5, 6, 7, 8, 9 or 10 nucleotides.

In a preferred embodiment, said loop region consists of 6, 7 or 9 nucleotides.

In some embodiments, the promoter of the hairpin-forming oligonucleotide may be any conventional procaryotic, eucaryotic, viral or synthetic promoter described in the art. Such a promoter may be ubiquitous or tissue-specific, homologous or heterologous to the template to be transcribed. Said promoter may be a wild type promoter or a variant with modified sequence.

In some embodiments, said promoter is a promoter of a bacteriophage, such as the bacteriophage T7, T3, Sp6, K11 or gh-1.

In some embodiments, the promoter is selected from T7 promoter, T3 promoter, Sp6 promoter, K11 promoter, gh-1 promoter and any variant thereof.

The term “T7 promoter”, as used herein, refers to the promoter of bacteriophage T7 having the sequence as set forth in SEQ ID NO: 1. The T7 promoter is specifically recognized by T7 RNA polymerase.

The term “T3 promoter”, as used herein, refers to the promoter of bacteriophage T3 having the sequence as set forth in SEQ ID NO: 2. The T3 promoter is specifically recognized by T3 RNA polymerase.

The term “Sp6 promoter”, as used herein, refers to the promoter of bacteriophage Sp6 having the sequence as set forth in SEQ ID NO: 3. The Sp6 promoter is specifically recognized by sp6 RNA polymerase.

The term “K11 promoter”, as used herein, refers to the promoter of bacteriophage K11 having the sequence as set forth in SEQ ID NO: 4. The K11 promoter is specifically recognized by K11 RNA polymerase.

The term “gh-1 promoter”, as used herein, refers to the promoter of bacteriophage gh-1 having the sequence as set forth in SEQ ID NO: 5. The gh-1 promoter is specifically recognized by gh-1 RNA polymerase.

The term “variant” of a given promoter, as used herein, refers to a promoter which is derived from a given promoter which is selected from T7 promoter, T3 promoter, Sp6 promoter, K11 promoter and gh-1 promoter and can be specifically recognized by the corresponding RNA polymerase for initiating the transcription. For example, a variant of T7 promoter is a promoter derived from the T7 promoter that can be specifically recognized by T7 RNA polymerase. Variants of above-mentioned promoters are well known in the art, e.g. described in Chen and Schneider, Nucleic Acids Res. 2005; 33(19):6172-6187. Or may be determined according to routine methods by the skilled person.

In a nucleic acid construct of the invention, the sequence of the reverse complement of a promoter is substantially reverse complementary to that of the promoter.

In some embodiments, the sequence of the reverse complement of a promoter is completely reverse complementary to that of the promoter.

In some embodiments, the sequence of the reverse complement of a promoter comprises from 1 to 10, particularly from 1 to 5, from 1 to 4, more particularly from 1 to 3 nucleotide deletions or substitutions compared to a sequence which is completely reverse complementary to that of the promoter.

### Use of the Nucleic Acid Construct

In some embodiments, the nucleic acid construct as defined above may be free in solution or be attached to a solid support by its 5′ end. Such solid support may be a resin, a bead, such as a magnetic bead, a capture bead or other materials (e.g., macro structures and/or insoluble materials) to form a capture bead, or a planar solid, such as a glass slide, a membrane, or a plate (e.g., a plate comprising multiple wells).

In some embodiments, the nucleic acid construct is covalently attached to a solid support, e.g. a resin by its 5′ end. In an embodiment, the nucleic acid construct is attached to a solid support by the 5′ end of the single-stranded nucleic acid. In another embodiment, said nucleic acid construct is attached to a solid support by a cleavable motif which can be cleaved to release the nucleic acid construct of the invention. The cleavable motif can be any means known in the prior art, such as a short oligonucleotide comprising at least one enzymatically cleavable nucleotide or a chemically cleavable internucleotide linkage. Examples of enzymatically cleavable nucleotides include, but not being limited to, deoxyuridine and deoxyinosine. For instance, the cleavable motif may be dIT. Example of chemically cleavable nucleotide is ribo-uracil (rU), which can be cleaved by KOH.

It is therefore an object of the present invention to provide a nucleic acid construct as defined above attached to a solid support by its 5′ end. In a particular embodiment, the nucleic acid construct is attached to the solid support by the termination motif at the 5′end of the nucleic acid construct. In another embodiment, the nucleic acid construct is attached to the solid support by a cleavable motif at the 5′end of the termination motif of the nucleic acid construct.

### Methods

The present invention also provides a method of generating an RNA product by using a nucleic acid construct as defined above. Said method comprises the steps of:
(a) providing a nucleic acid construct as defined above,
(b) contacting the nucleic acid construct with an RNA polymerase and ribonucleotides to produce the RNA product.

### Step (a)

In some embodiments, the nucleic acid construct used in step (a) is free in solution.

In some embodiments, the nucleic acid construct used in step (a) is attached to a solid support by its 5′ end.

Thanks to its primary single-stranded structure, the nucleic acid construct of the invention used in step (a) may be synthesized easily by any conventional method, for example by chemical synthesis based on solid-phase phosphoramidite chemistry described by Adams et al. (1983, J. Amer. Chem. Soc., 105, 661) and Froehler et al. (1983, Tetrahedron Lett., 24, 3171) or by enzymatic synthesis. Template-independent enzymatic polynucleotide synthesis methods are for example described in detail in WO 2015/159023, WO 2017/216472, U.S. Pat. Nos. 5,436,143, 5,763,594, Jensen et al (Biochemistry, 57:1821-1832 (2018)), or Mathews et al (Organic & Biomolecular Chemistry, DOI: 0.1039/c6ob01371f (2016)); Schmitz et al (Organic Lett., 1(11):1729-1731 (1999)).

In some embodiments, the method of the invention comprises a step of synthetizing the nucleic acid construct before step (a).

In some embodiments, the step of synthetizing the nucleic acid construct is performed by an enzymatic method, particularly an enzymatic method using template-free polymerases, such as terminal deoxynucleotidyl transferase (TdT) or variants such as those described in detail in WO2019/135007 for DNA synthesis or a polyA polymerase (PAP) or polyU polymerase (PUP) or variant thereof usually for RNA synthesis (e.g. Heinisch et al, WO2021/018919). In some embodiments, the nucleic acid construct is synthesized from an initiator having a free 3′-hydroxyl. Said initiator may be attached by its 5′ end to a solid support, by any conventional methods.

In some embodiments, said enzymatic method comprises the step of (i) contacting an initiator or an elongated fragment thereof having a free 3′-hydroxyl with a 3′-O-blocked nucleoside triphosphate and a template-free polymerase to form a 3′-O-blocked elongated fragment, (ii) deblocking the elongated fragments to form an elongated fragment having free 3′-hydroxyl, and (iii) repeating cycles of (i) and (ii) until the nucleic acid construct is formed.

### Step (b)

By “contacting”, it is meant that an RNA polymerase is added to a reaction medium containing a nucleic acid construct under reactional conditions suitable for in vitro transcription. These conditions are well known to a skilled person (Jani and Fuchs, J Vis Exp. 2012 Mar. 26 (61): 3702). At the end of step (b), the template part in the nucleic acid construct is transcribed by the RNA polymerase and an RNA product is generated and released in the reaction medium.

The reaction medium advantageously contains the four natural ribonucleotides (rNTP), e.g. rATP, rUTP, rGTP, rCTP. The reaction medium might also contain non-natural nucleotides which may be incorporated into the RNA product during the in vitro transcription by the RNA polymerase. Non-natural nucleotides may be present in the reaction medium at various ratios vs the natural rNTP. The non-natural nucleotides in the reaction medium may be modified nucleotides comprising a reactive group for a “click” reaction, e.g. 5-octynyl-rNTPs, which may be added randomly in the RNA during the in vitro transcription. The non-natural nucleotides include but are not limited to 2′-O-methyl modified nucleotides, 2′-O-methoxy-ethyl modified nucleotides 2′-F modified nucleotides, alpha-phosphothioate modified nucleotides, which increase the stability against an RNA degradation enzyme (RNase) of an RNA product generated by in vitro transcription (IVT) and increase the affinity of the said RNA product for in vivo DNA or RNA targets. Other non-natural nucleotides may be 5′caps, e.g. m7GpppG, m7GpppA, G5ppp5A, G5ppp5G, 3′-O-Me-m7G (5′) ppp (5′) G, and derivatives thereof, which are incorporated selectively at the 5′end of an RNA product by an RNA polymerase. The 5′cap increases the stability against nucleases and reduces toxic side effects of the RNA in vivo due to the absence of the 5′-triphosphate group. Variants of m7GpppG include but are not limited to: i) biotinylated-m7GpppG which allows the RNA to be used as a capture probe, e.g. target enrichment, particularly in NGS target enrichment, ii) fluorescent-m7GpppG which allows the RNA to be used in imaging applications in vivo (e.g. FISH probe), iii) alkyne-m7GpppG which allows the post-transcriptional chemical modification of the RNA by copper-catalyzed click reaction. Similar variants of other 5′caps are also described in the prior art.

Optionally, the method of the invention may further comprise a step (c) which consists of one or more repetitions of step (b) to produce several copies of said RNA product.

According to the present invention, the RNA polymerase hybridizes directly to the nucleic acid construct to synthesize an RNA product, i.e., the method is free of any additional intermediary step, wherein the nucleic acid construct would hybridize to a complementary DNA fragment.

Therefore, in a preferred embodiment, the method consists of the steps:
(a) providing a nucleic acid construct as defined above, and
(b) contacting the nucleic acid construct with an RNA polymerase and ribonucleotides to produce the RNA product, and optionally
(c) repeating step (b) to produce several copies of said RNA product.

Advantageously, the nucleic acid of step (a) is attached to a solid support.

The choice of the RNA polymerase is adapted to the promoter in the nucleic acid construct. For examples, if the promoter in the nucleic acid construct is T7 promoter or a variant thereof, T7 RNA polymerase will be used in step (b). According to the selected RNA polymerase, the person skilled in the art is able to adapt the reactional conditions (e.g. pH and temperature) of step (b).

### Optional Steps (d) and (e)

The RNA product generated at step (b) or step (c) may be further submitted to at least one post-transcriptional modification (step (d)).

The term “post-transcriptional modification” refers to biological processes by which a primary RNA is chemically altered to produce a mature, functional RNA molecule. The term “primary RNA” refers to the RNA directly generated after the transcription. In the context of the present invention, the RNA generated at step (b) or step (c) is a primary RNA. The skilled person is able to determine if at least one post-transcriptional modification is necessary for an RNA product obtained at step (b) or step (c) according to the intended use of said RNA product. For instance, if a primary RNA is produced to be further used for protein translation, then at least one post-transcriptional modification might be necessary to convert said primary RNA to a mature mRNA. Generally speaking, post-transcriptional modifications comprise modifications concerning the 5′ end and/or the 3′ end of the primary RNA. Examples of post-transcriptional modifications include, without limitations, 5′ capping, 3′ extension after transcription, and intron splicing.

In some embodiments, the at least one post-transcriptional modification is a 5′ capping, a 3′ extension with a polyA polymerase (PAP), ligations of modified polyA tail or a chemical modification of a non-natural nucleotide using a “click” reaction.

The term “5′ capping” refers to the addition of a 5′cap as described above to the 5′ end of a primary RNA.

The term “3′ extension with a polyA polymerase” refers to the addition of adenines at the 3′end of a primary RNA to form a polyA tail.

The term “ligations of modified polyA tail” refers to the chemical or enzymatic addition of a modified polyA tail (e.g. containing 2′-O-methyl modified nucleotides or 2′-F-modified nucleotides) to the 3′end of the primary RNA. Chemical ligation can be performed by a cycloaddition reaction (e.g. a “click” reaction), a nucleophilic substitution (e.g. NHS ester substitution) or other click reactions. Enzymatic ligation can be performed by using a ligase for adding a modified polyA tail.

Examples of chemical modifications of non-natural nucleotides may be copper-catalyzed or strain-promoted “click reaction”, nucleophilic substitution reaction, cycloaddition reactions, or metathesis reaction. For example, a primary RNA comprising a non-natural nucleotide containing an alkyne group, e.g. 5-octynyl-rNTP or alkyne-m7GPPPG, may perform a post-transcriptional chemical copper-catalyzed or strain-promoted “click reaction” with an azide-modified functional molecule. Examples of said functional molecule may be biotin (e.g. PEG4 carboxamide-6-Azidohexanyl Biotin), a fluorophore (e.g. 5-FAM-azide), protein label (e.g. antibody-azide), or a drug payload (e.g. artemisinine-azide).

The in vitro methods for implementing at least one of such post-transcriptional modifications are well described in the art (Warren, L., et al. (2010) Cell Stem Cell, 7, 618-630). A skilled person is able to implement the dedicated method according to the modifications to be made.

The method of the invention may further comprise a step (e) for recovering the RNA product generated in steps (b), (c) and/or (d). This step permits to separate the RNA product from the nucleic acid construct and the reaction medium.

The recovery step may be performed by any standard nucleic acid purification method, for example by gel purification, column of affinity, or any commercially available nucleic acid purification kit.

In a particular embodiment, the method comprises or consists of the steps:
(a) providing a nucleic acid construct as defined above, preferably attached to a solid support, and
(b) contacting the nucleic acid construct with an RNA polymerase and ribonucleotides to produce the RNA product, and optionally
(c) repeating step (b) to produce several copies of said RNA product, and/or optionally
(d) implementing at least one post-transcriptional modification, and/or optionally
(e) recovering said RNA product produced in steps (b), (c) and/or (d).

### Kits

The present invention also provides kits for generating an RNA product. The term “Kit” refers to any necessary reagents, and optionally means, for generating an RNA product by use of a nucleic acid construct or a method of the invention.

In some embodiments, the kit of the invention comprises an RNA polymerase, several ribonucleotides, one or more reaction buffers and a nucleic acid construct as defined above.

The RNA polymerase comprised in the kit may be any conventional procaryotic, eucaryotic or viral RNA polymerase as described in the art. In a particular embodiment, the RNA polymerase is an RNA polymerase of a bacterium or a bacteriophage. In a more particular embodiment, the RNA polymerase of the kit is selected from T7 RNA polymerase, T3 RNA polymerase, SP6 RNA polymerase, K11 RNA polymerase and gh-1 RNA polymerase.

In some embodiments, the kit comprises an RNA polymerase which specifically recognizes the promoter in the nucleic acid construct of the kit. In a particular embodiment, the kit comprises an RNA polymerase which is homologous with respect to the promoter comprised in the nucleic acid construct of the kit, as described above.

The ribonucleotides comprised in the kit are natural ribonucleotides, i.e. adenosine, cytidine, guanosine, and uridine.

The kit of the invention may further comprise non-natural nucleotides as described above, which may be incorporated into an RNA product during an in vitro transcription by an RNA polymerase.

The reaction buffer(s) may contain salt and any reagents to optimize the activity of the enzymes comprised in the kit.

Alternatively, the kit of the invention comprises any necessary reagents for synthesizing a nucleic acid construct as defined above, as well as the reagents necessary to generate an RNA product from said nucleic acid construct. In such an embodiment, the kit comprises an RNA polymerase, several ribonucleotides and optionally non-natural nucleotides, one or more reaction buffers, an initiator having a 3′-terminal nucleotide having a free 3′hydroxyl, a template-free polymerase, a plurality of 3′-O-blocked nucleoside triphosphates and a deblocking agent. In context of the present invention, the term “initiator” refers to a short single-stranded oligonucleotide with a free 3′-hydroxyl, which can be further elongated by a template-free polymerase, such as a TdT. The initiator may comprise between 3 and 100 nucleotides, in particular between 3 and 20 nucleotides. In some embodiments, the initiator may comprise a non-nucleic acid compound having a free hydroxyl to which a TdT may couple a 3′-O-blocked dNTP.

The template-free polymerase may be any template-free DNA polymerase, such as a TdT or variant thereof usually for DNA synthesis (e.g. Ybert et al, WO2017/216472; Champion et al, WO2019/135007). Or a polyA polymerase (PAP) or polyU polymerase (PUP) or variant thereof usually for RNA synthesis (e.g. Heinisch et al, WO2021/018919).

The 3′-O-blocked nucleoside triphosphates may be 3′-O-blocked-dNTP and/or 3′-O-blocked-rNTP. These compounds comprise a blocking group which protects the hydroxyl group at the 3′position from undergoing a chemical change during a chemical or enzymatic process. This blocking group may be any blocking group known in the prior art, e.g. 3′-O—NH₂, 3′-O-azidomethyl, 3′-O-allyl, or 3′-O-phosphate.

Accordingly, the kit of the invention may also comprise a “de-blocking agent” which is a chemical or enzymatic agent able to cleave a special blocking group. The selection of deblocking agent depends on the type of 3′-nucleotide blocking group used, whether initiators are attached to solid supports, and the like. For example, a phosphine, such as tris(2-carboxyethyl)phosphine (TCEP) can be used to cleave a 3′O-azidomethyl group, palladium complexes can be used to cleave a 3′O-allyl group, or sodium nitrite can be used to cleave a 3′O-amino group.

In some embodiments, the kit further comprises a solid support, wherein the initiator or the nucleic acid construct is attached to a solid support by its 5′ end. The solid support may be a resin, a bead or a plate.

The kits of the invention may further comprise a pyrophosphatase. Said pyrophosphatase may be any conventional pyrophosphatase for in vitro transcription, e.g. inorganic pyrophosphatase. Said enzyme catalyzes the hydrolysis of inorganic pyrophosphate and can be used for the enhancement of RNA/DNA synthesis because inorganic pyrophosphate is a side product on nucleotide addition and can prevent the addition of a subsequent nucleotide.

The kit of the invention may further comprise the reagents necessary for implementing at least one post-transcriptional modification on the generated RNA. These reagents may be the chemical compounds and/or enzymes necessary for implementing a 5′ capping, a 3′ extension and/or ligations of modified polyA tail. For instance, these reagents may be a polyA polymerase (PAP), T7 ligase, or the chemical compounds for performing a cycloaddition reaction.

The kit of the invention may further comprise any delivery system for delivering materials. Such delivery systems include systems for the storage, transport, or delivery of reaction reagents from one location to another, and/or supporting materials (e.g., reaction medium, written instructions for performing the assay etc.). For example, kits may include one or more enclosures (e.g., boxes) containing the relevant reaction reagents and/or supporting materials.

The kits of the invention may also comprise means for recovering the generated RNA. Such means may be any conventional polynucleotide recovery systems, such as a column for purification by affinity or by precipitation.

In some embodiments, the kit of the invention is a kit for generating a sgRNA. Said kit comprises an RNA polymerase, several ribonucleotides, an initiator having a 3′-terminal nucleotide having a free 3′hydroxyl, a template-free DNA polymerase, a plurality of 3′-O-blocked nucleoside triphosphates and a deblocking agent, wherein the initiator comprises a deoxyribonucleic acid having the complementary sequence of the constant part of a sgRNA.

Alternatively, the kit for generating a sgRNA comprises an RNA polymerase, several ribonucleotides and a nucleic acid construct of the invention comprising a single-stranded nucleic acid comprising a template part for generating a sgRNA.

The present invention is illustrated more in detail by following examples.

### EXAMPLES

### In Vitro Transcription

In vitro transcription was implemented in a microplate in a reaction medium of 30 μl containing 6.7 mM NTP Buffer Mix, 1 μM free nucleic acid construct, 60 μM DFHBI-1T, 2 μl T7 RNA polymerase Mix (HiScribe™ T7 High Yield RNA Synthesis Kit from NEB) and 14 μl nuclease-free water. The reaction medium for immobilized constructs is the same with the exception that the medium comprises 7.5 μM immobilized nucleic acid in place of 1 μM free nucleic acid construct. The microplate was then incubated at 37° C. and the fluorescence was read in GFP channel (excitation at 395 nm and detection at 509 nm) for 18 hours with agitation.

Fluorescence Monitoring by qPCR

In vitro transcription was implemented according to the method described above, with the difference that the microplate was incubated at 37° C. without agitation and the fluorescence was read in SYBR channel (excitation from 450-490 nm and detection from 515-530 nm) for 12 hours.

### Gel Electrophoresis of Transcription Products

After in vitro transcription, 5 μL of reaction medium and 5 μL of RNA loading dye were mixed. The mixture was boiled at 98° C. for 5 min. After cooling on ice, the mixture was loaded in a TBE-Urea 10% gel (Novex™ TBE-Urea Gels, Invitrogen™). Said gel was prepared with high purity reagents including Tris Base, Boric Acid, EDTA, Acrylamide, Bisacrylamide, TEMED, APS and 7M Urea. A gel electrophoresis was run at 180 V during 1 hour.

### Example 1: In Vitro Transcription from Free Nucleic Acid Constructs

Five nucleic acid constructs, including two constructs of the invention, i.e. “6-nt-loop”, “9-nt-loop”, two positive control constructs (“dsDNA” and “ssDNA+promoter”) and a negative control “ssDNA” were provided as free nucleic acid constructs in solution.

The constructs of the invention comprise sequentially, from 5′ to 3′ end, a single-stranded DNA consisting of the template part of Broccoli RNA to be synthesized, the reverse complement of T7 promoter, a loop of 6 or 9 nucleotides, and T7 promoter. The constructs “6-nt-loop” and “9-nt-loop” have respectively the sequences of SEQ ID NO: 6 and SEQ ID NO: 7.

The construct “dsDNA” comprises a double-stranded DNA encoding Broccoli RNA and a double-stranded functional T7 promoter. Said construct is formed by a forward DNA strand of sequence SEQ ID NO: 8 and a reversed DNA strand of sequence SEQ ID NO: 9.

The construct “ssDNA+promoter” comprises a single-stranded DNA as the template of Broccoli RNA and a double-stranded functional T7 promoter. Said construct is formed by a forward strand of sequence SEQ ID NO: 1 and a reversed strand of sequence SEQ ID NO: 9.

The negative control “ssDNA” is a single-stranded DNA comprising sequentially the template of Broccoli RNA and T7 promoter. Said construct has the sequence of SEQ ID NO: 9.

Broccoli RNA is a small RNA aptamer which can bind to a small molecule DFHBI-1T and becomes fluorescent like the GFP (Milligan et al., Nucleic Acids Res. 1987 Nov. 11; 15(21):8783-98). Thereby, the quantity of Broccoli RNAs generated from these free nucleic acid constructs by in vitro transcription can be monitored by quantitative fluorescence emission measured by a qPCR instrument.

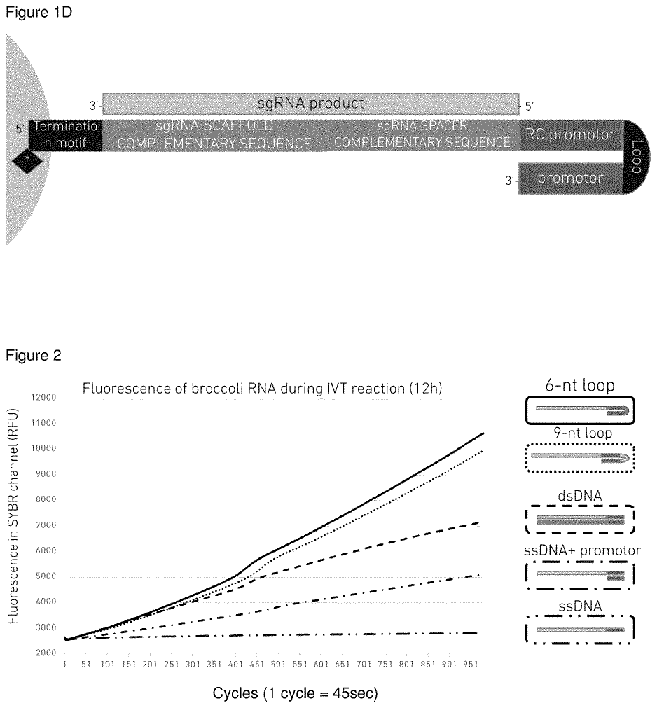

In vitro transcription was performed at 37° C. for 12 hours. The fluorescence intensity is correlated to the quantity of Broccoli RNA generated and was measured each 90 seconds by a qPCR instrument (CFX96 real-time system C1000 Touch thermal cycler from Bio-Rad) using the SYBR channel (excitation at 395 nm and detection at 509 nm). The results are shown in FIG. 2. Both tested free nucleic acid constructs of the invention allowed to generate Broccoli RNA more quickly than the positive control constructs “dsDNA” or “ssDNA+promoter”. Therefore the nucleic acid constructs of the invention are more efficient than the positive control constructs in producing RNAs.

### Example 2: In Vitro Transcription from Immobilized Nucleic Acid Constructs

Two nucleic acid constructs of the invention were immobilized on resin (“immobilized nucleic acid constructs”), i.e. “Resin 6-nt loop” and “Resin 9-nt loop”. Said constructs were linked to the resin by the 5′ end and comprise sequentially, from 5′ to 3′ end, an inosine, a single-stranded DNA consisting of the template part of Broccoli RNA, the reverse complement of T7 promoter, a loop of 6 or 9 nucleotides, and T7 promoter. The constructs “Resin 6-nt loop” and “Resin 9-nt loop” have respectively the sequences SEQ ID NO: 10 and SEQ ID NO: 11.

Said immobilized constructs and the free constructs “9-nd loop”, “6-nd loop”, “dsDNA” and “ssDNA” as described in Example 1 were used for in vitro transcription.

In vitro transcription was performed at 37° C. during 17.5 hours. The fluorescence intensity was measured each 90 seconds by a qPCR instrument (CFX96 real-time system C1000 Touch thermal cycler from Bio-Rad) using the SYBR channel.

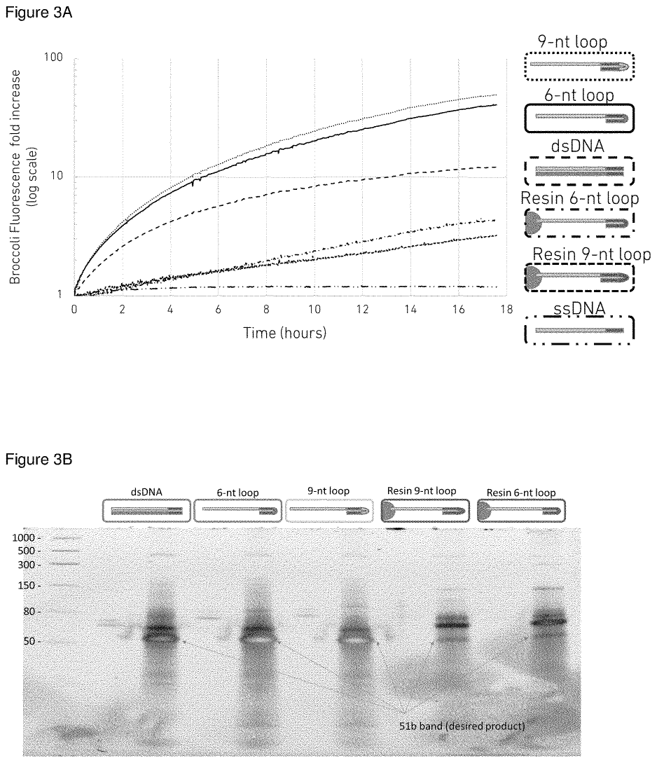

The results of fluorescence intensity show that immobilized nucleic acid construct “Resin 6-nt loop” and “Resin 9-nt loop” can efficiently produce Broccoli RNA (FIG. 3A). FIG. 3B also confirms that the RNA product obtained is Broccoli RNA (51 bp).

### Example 3: Efficiency of Termination Motifs for Stopping In Vitro Transcription

In vitro transcription (IVT) was performed by using nucleic acid constructs of the invention. Said constructs comprise a short single-stranded nucleic acid which is composed of, from 5′ to 3′, a sequence of 15 nucleotides, a termination motif, and a sequence of 21 nucleotides as the template part for generating an RNA. The termination motif is positioned between the two sequences and at the 5′ end of the sequence of 21 nucleotides. The termination motifs are two adjacent abasic sites (AP2), three adjacent abasic sites (AP3), two abasic sites which are separated by a deoxyinosine (APiAP), two abasic sites which are separated by a deoxythymine (APTAP), three abasic sites each being separated by a deoxythymine (APT3) and a triethylene glycol spacer (C9S). In vitro transcription was also performed by using the constructs “IVT Stop” and “No stop” as control. The construct “No stop” comprises a template of 36 nucleotides devoid of a termination motif between the sequence of 21 nucleotides and the sequence of 15 nucleotides. The construct “IVT Stop” comprises a template of 21 nucleotides.

RNA products generated after IVT were analyzed on TBE-Urea 15% gels (Novex™ TBE-Urea Gels, Invitrogen™).

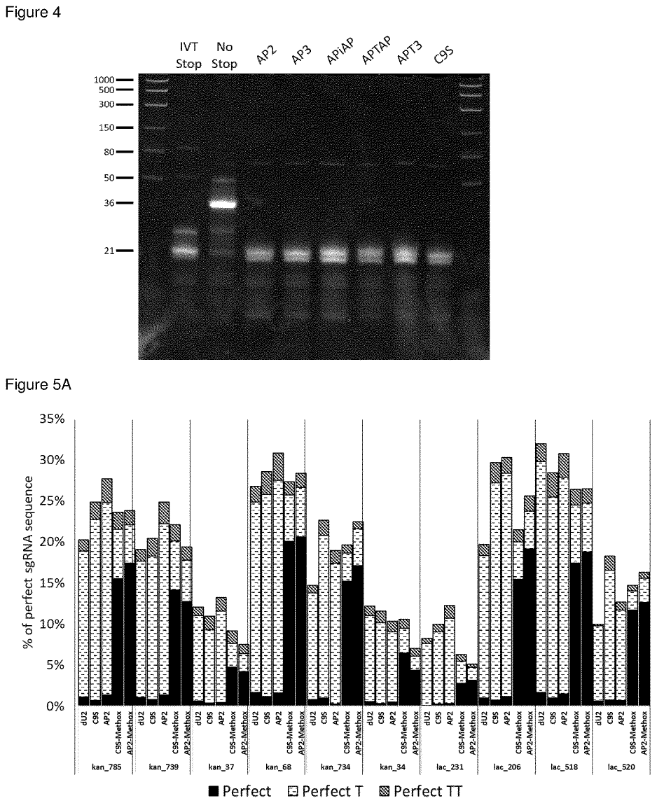

FIG. 4 shows that the constructs comprising the termination motifs AP2, AP3, ApiAP, APTAP, APT3 and C9S all generate an RNA of 21 nucleotides, which indicates that these termination motifs are efficient to stop the transcription.

### Example 4: Effect of 2′-O-Methoxy-Ethyl Modified Nucleotides in Nucleic Acid Constructs of the Invention

During IVT, undesirable additional dT may be added by the RNA polymerase to an RNA generated from its template. Those additional dT are the consequence of the RNA polymerase untemplated activity. To assess the effect of the 2′-O-methoxy-ethyl group on the activity of RNA polymerase, 10 single guide RNAs (sgRNA) were synthesized from different nucleic acid constructs of the invention by use of the T7 RNA polymerase. These constructs comprise a template part for generating a sgRNA and a termination motif at the 5′ end of the template part. The tested termination motif is either AP2, C9S, dU2, AP2 combined with a 2′-O methoxy-ethyl modified nucleotide (“AP2-Methox”) or C9S combined with a 2′-O methoxy-ethyl modified nucleotide (“C9S-Methox).

Before proceeding with IVT, the nucleic acid constructs comprising dU2 were incubated for 1 h at 37° C. with UDG enzyme (NEB) to convert deoxyuridin (dU) into an abasic site.

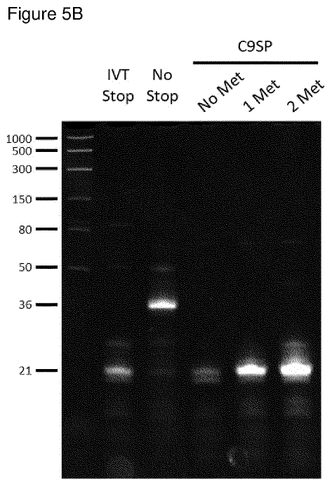

After in vitro transcription, the generated sgRNAs were submitted to RNA sequencing. The library preparation was performed using the SEQuoia Complete Stranded RNA Library Prep Kit (Bio-Rad). FIG. 5A shows that, compared to AP2, C9S or dU2, a termination motif comprising a 2′-O-Methoxy-ethyl modified nucleotide (“C9S-Methox” and “AP2-Methox”) dramatically reduces the T7 polymerase untemplated activity.

In addition, the effect of the 2′-O-Methoxy-ethyl modified nucleotide was tested on single-stranded nucleic acids composed of, from 5′ to 3′, a sequence of 15 nucleotides, a termination motif, and a sequence of 21 nucleotides as the template part of the single-stranded nucleic acid. The termination motif is C9SP ((No Met) or C9SP combined with one (1Met) or two (2 Met) 2′-O-methoxy-ethyl modified nucleotides. After IVT, generated RNA products were analyzed on TBE-Urea 15% gels (Novex™ TBE-Urea Gels, Invitrogen™). FIG. 5B shows that the presence of one or two 2′-O-methoxy-ethyl modified nucleotides in a termination motif increases the RNA yield from the templates.

Therefore, a termination motif comprising one or two 2′-O-methoxy-ethyl modified nucleotides in a nucleic acid construct of the invention reduces the untemplated activity of an RNA polymerase and increases the yields of RNA generated by an in vitro transcription.

1. A nucleic acid construct for generating at least one RNA product, comprising, from 5′ to 3′:
(i) a single-stranded nucleic acid comprising at least one template part for generating the RNA product, and
(ii) a hairpin-forming oligonucleotide which comprises successively a reverse complement of a promoter, a loop region and the promoter, said reverse complement of a promoter and said promoter being capable of forming a double-stranded oligonucleotide,
wherein the single-stranded nucleic acid comprises at least one termination motif at the 5′ end of the template part of the single-stranded deoxyribonucleic acid, wherein said termination motif comprises at least two components selected from modified nucleotides, abasic sites, modified abasic sites, hairpin structures, protein binding sites, chemical linkers and combinations thereof.

2. The nucleic acid construct according to claim 1, wherein the termination motif comprises (i) at least two modified nucleotides or (ii) at least two abasic sites or modified abasic sites.

3. The nucleic acid construct according to claim 2, wherein the termination motif further comprises one or more chemical linkers.

4. The nucleic acid construct of claim 1, wherein said promoter is a T7 promoter, a T3 promoter, an Sp6 promoter, a K11 promoter, or a gh-1 promoter, or variants thereof.

5. The nucleic acid construct according to claim 1, wherein said single-stranded nucleic acid comprises a template part for generating a sgRNA, a small RNA, an RNA aptamer or a mRNA.

6. The nucleic acid construct of claim 1, wherein said loop region comprises at least 3 nucleotides.

7. The nucleic acid construct according to claim 6, wherein said loop region comprises 6, 7 or 9 nucleotides.

8. The nucleic acid construct of claim 1, wherein said single-stranded nucleic acid has a length of at least 10 nucleotides.

9. The nucleic acid construct of claim 1, wherein said nucleic acid construct is attached to a solid support by a 5′ end of the nucleic acid construct.

10. A kit for generating an RNA product, comprising:
an RNA polymerase, a plurality of ribonucleotides, one or more reaction buffers, and
(i) the nucleic acid construct of claim 1, or (ii) an initiator having
a 3′-terminal nucleotide having a free 3′-hydroxyl, a template-free DNA polymerase, a plurality of 3′-O-blocked nucleoside triphosphates, and a deblocking agent.

11. The kit according to claim 10, further comprising a solid support, wherein the nucleic acid construct or the initiator is attached to the solid support by a 5′ end of the nucleic acid construct or the initiator.

12. The kit according to claim 10, wherein the kit is for generating a sgRNA, said kit comprising an initiator having a deoxyribonucleic acid sequence complementary to a nucleic acid sequence of a constant part of said sgRNA.

13. A method of generating an RNA product, comprising the steps of:
(a) providing a nucleic acid construct according to claim 1, and
(b) contacting the nucleic acid construct with an RNA polymerase and ribonucleotides to produce the RNA product.

14. The method according to claim 13, comprising a step of synthetizing the nucleic acid construct before step (a).

15. The method according to claim 14, wherein the step of synthetizing the nucleic acid construct is performed by an enzymatic method.

16. The method of claim 13, wherein the nucleic acid construct of step (a) is attached to a solid support by a 5′ end of the nucleic acid construct.

17. The method of claim 13, wherein the nucleic acid construct comprises a termination motif comprising (i) at least two modified nucleotides and (ii) one or more chemical linkers.

18. The method of claim 13, wherein the nucleic acid construct of step (a) comprises a template part for generating a sgRNA.

19. The method of claim 13, further comprising one or more of:
(c) repeating step (b)
(d) implementing at least one post-translational modification, and
(e) recovering the RNA product produced in step (b), (c), or (d).
